(19) United States
(12) Patent Application Publication
(10) Pub. No.: US 2013/0012828 A1
(43) Pub. Date: Jan. 10, 2013

(54) Method and System for Measuring Nasal Resistance to Airflow

(75) Inventor: Alonzo C. AYLSWORTH, Wildwood, MO (US)

(73) Assignee: 3 INVENTING GUYS, LLC, Austin, TX (US)

(21) Appl. No.: 13/448,510

(22) Filed: Apr. 17, 2012

Related U.S. Application Data
(60) Provisional application No. 61/506,030, filed on Jul. 9, 2011.

(51) Int. Cl. A61B 5/087

(52) U.S. Cl. 600/538

(57) ABSTRACT

Determining nasal resistance. At least some of the illustrative embodiments are methods including: applying negative pressure to a patient's mouth and thereby creating airflow that flows in a first nare and out the patient's mouth; measuring at least one property of the airflow during the causing; and determining an indicia of nasal resistance of the patient, the determining based on the at least one property.

### CROSS-REFERENCE TO RELATED APPLICATIONS

This application claims benefit of U.S. Provisional Application No. 61/506,030 filed 9 Jul. 2011 titled “Method and System for Measuring Nasal Resistance to Air Flow,” the disclosure of which is incorporated herein by reference as if reproduced in full below. Moreover, this application is related to U.S. application Ser. No. 13/220,341 filed 29 Aug. 2011 titled “Method and System for Measuring Nasal Resistance to Airflow”

### BACKGROUND

Knowing a patient's nasal resistance to airflow may be useful in predicting and diagnosing many ailments associated with the nose, nasopharynx, and velum (e.g., a deviated septum, or a sleep disorder such as Obstructive Sleep Apnea (OSA)). Rhinomanometry is one technique of determining nasal resistance to airflow that includes intentionally blocking a patient's nare, covering the patient's mouth, and simultaneously recording flow through the unblocked nare in relation to the pressure developed in the blocked nare. The process is then reversed and the nasal resistance to airflow of the second nare determined.

As can be appreciated, a rhinomanometric test may be a complicated and uncomfortable process for the patient. Additionally, rhinomanometry may have a number of errors associated with the processes. For example, the airflow and pressure is dependent on the patient's respiratory effort, which may be inconsistent breath-to-breath. Additionally, the airflow through each nare is measured with the other nare blocked, and therefore the nasal resistance calculated is not with respect to normal breathing patterns. Further, the act of plugging a nare, so as to read nasal pressure, may cause swelling of the nasal tissue which may in turn affect airflow through that nare and skew the calculated nasal resistance. Further complicating the measurement, the patient's soft palate may fully or partially block a pneumatic connection to the lungs. Inaccurate readings and potentially a misdiagnosis of patient nasal resistance may result.

### BRIEF DESCRIPTION OF THE DRAWINGS

For a detailed description of various embodiments, reference will now be made to the accompanying drawings in which:

FIG. 1 illustrates a block diagram of a system for determining nasal resistance according various embodiments of the present disclosure;

FIG. 2 illustrates a system for determining nasal resistance in fluid communication with a patient (shown in cross-section) according various embodiments of the present disclosure;

FIG. 3 illustrates a method for determining nasal resistance according to various embodiments of the present disclosure;

FIG. 4 illustrates a method for determining nasal resistance according to another embodiment of the present disclosure;

FIG. 5 illustrates a method for determining nasal resistance according to another embodiment of the present disclosure;

FIG. 6 illustrates a method for determining nasal resistance according to another embodiment of the present disclosure;

FIG. 7 illustrates a block diagram of a system for determining nasal resistance according other embodiments of the present disclosure;

FIG. 8 illustrates a method for determining nasal resistance according to various embodiments of the present disclosure;

FIG. 9 illustrates a method for determining nasal resistance according to another embodiment of the present disclosure;

FIG. 10 illustrates a method for determining nasal resistance according to another embodiment of the present disclosure; and

FIG. 11 illustrates a method for determining nasal resistance according to another embodiment of the present disclosure.

### NOTATION AND NOMENCLATURE

Certain terms are used throughout the following description and claims to refer to particular system components. As one skilled in the art will appreciate, different companies may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

“About” in reference to a value shall mean the recited value and within fifteen percent (15%) of the recited value.

“Airflow sensor” shall mean a sensor that measures airflow. A pressure sensor shall not be considered an “airflow sensor.”

“Pressure”, with respect to applying pressure to the nares or mouth of a patient, shall be in reference to gauge pressure such that “positive pressure” shall be a pressure above atmospheric pressure, and “negative pressure” shall be a pressure below atmospheric pressure.

### DETAILED DESCRIPTION

Overview:

As noted herein, rhinomanometric testing for nasal resistance may cause discomfort for the patient and further, may have many potential sources of error. As such, the present disclosure is drawn to a method and system for determining nasal resistance that may improve the comfort for the patient and reduce the potential errors in measurement of nasal resistance.

In the present disclosure, the method and system provide indicia of nasal resistance. Further, the method and system are capable providing indicia of nasal resistance from compliant patients (i.e., a patient who will follow directions regarding temporary cessation of respiration), as well as non-compliant patients (i.e., those that cannot or will not follow directions regarding temporary cessation of respiration). Further, the present disclosure relates to causing airflow into the nares or nostrils of a patient by a motive force outside of the patient's body or physiological efforts. Thusly, the somewhat uncontrolled variable of a patient's inspiratory effort may be removed from the determinations of nasal resistance. In some embodiments, positive pressure is applied to the patient to cause airflow, and in other embodiments negative pressure is applied to cause airflow.

The system comprises a plurality of interfaces with the patient's breathing orifices, particularly the nares and mouth, and the interfaces are configured in some cases to be positioned externally (e.g., nasal pillows), and in other cases the interfaces may seal to an inside surface of the patient's breathing orifice (e.g., mouth). Further, the system comprises a pressure and/or vacuum source in fluid communication with the interfaces and configured for control of airflow. The pressure and/or vacuum source is controlled by a processor in operative communication with a plurality of sensors. The processor alters the operation of the pressure and/or vacuum source to the interfaces by opening or closing certain valves disposed between the source and the interfaces, and/or controlling the source itself. The processor alters the operation to control airflow amount and direction through the interfaces in response to instructions stored on a memory device.

The system of the current disclosure is configured in some cases as a standalone device. The system may be considered a diagnostic device, such as a nasal function test device, and thus the system is not a device for providing therapeutic positive airway pressure to treat disordered breathing, such as a Continuous Positive Airway Pressure (CPAP) or bi-level PAP (BIPAP) device. The description first focuses on devices that apply positive pressure to induce airflow. Devices that apply negative pressure or vacuum are discussed later in the specification.

One method for determining nasal resistance is airflow from the pressure source is directed to each of the patient's nares via the interfaces. In certain instances, airflow is directed to the patient's mouth. The airflow may be introduced to each nare simultaneously or separately. The airflow to each nare may have a measureable property, such as velocity (i.e. rate), pressure, or volume. In many cases, the airflow may be introduced to each nare such that a portion of the airflow exits or escapes the patient's mouth. Various properties of the airflow introduced to the nares or the airflow from the mouth may be measured. The measured properties are then compared and analyzed by the processor according to instructions stored on a memory device.

Some embodiments of the method also include adjusting the introduced airflow in response to the measurements taken at the mouth and/or nares. More specifically, the measurements taken at the mouth or nare may serve to coordinate certain properties of the introduced airflow with the patient's respiration. The measurements related to the mouth and each nare are compared by the processor and adjustments are made according to instructions stored on the memory device.

System:

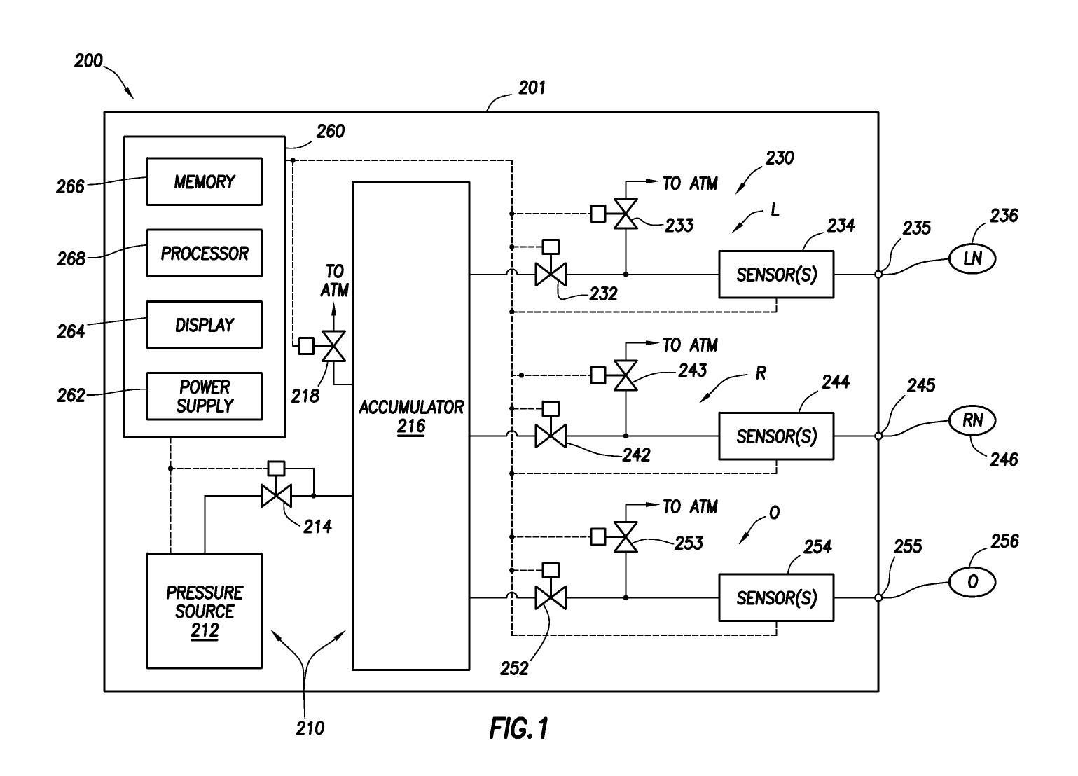

Referring now to FIG. 1, which illustrates a system 200 that operates based on application of positive pressure to one or more of the patient's breathing orifices. The system 200 comprises both electrical connections and fluid connections. For purposes of clarity, in FIG. 1 electrical connections will be shown with dashed lines, and fluid connections with solid lines.

The system 200 includes a case or cover 201 containing an airflow subsystem 210, a distribution subsystem 230, and a control subsystem 260. The airflow subsystem 210 is in fluid communication with the distribution subsystem 230. The control subsystem 260 is in electrical communication with the airflow subsystem 210 and the distribution subsystem 230. The system 200 is configured to provide positive pressure from the airflow subsystem 210 to a patient via the distribution subsystem 230 in response to commands from the control subsystem 260.

According to these embodiments, the airflow subsystem 210 may have airflow source 212, airflow control 214, and accumulator or airflow storage 216. The airflow source 212 is a device configured to produce airflow for passage through the system 200. In non-limiting examples, the airflow source 212 may be a compressor, a fan, a pump, a bellows, or any other device configurable to produce airflow. In some instances, the airflow source 212 may be generically referred to as a blower, such as variable speed blowers that find use in CPAP machines. In further instances, the airflow source 212 produces airflow from environmental air, compressed air (i.e., from a storage vessel), a mixed gas air from purified storage vessels, or some combination thereof.

The airflow control 214 is a device configured to change the properties of airflow through the system 200. In non-limiting examples, the airflow control 214 may be a valve, an actuator, an orifice, a conduit, or other device configurable to stop, redirect, control, or otherwise regulate the airflow from airflow source 212 through the system 200.

Airflow storage 216 is a device configurable to retain airflow (i.e., gas) between airflow source 212 and the rest of the system 200. The airflow storage 216 is a device configured to retain or store airflow at predetermined properties, such as but not limited to pressure, temperature, gas mixture, humidity, or some combination thereof. The airflow storage 216 may be an accumulator, a pressure cylinder, a vessel, a collapsible bag, or other device configurable for retaining a gas such as air for an indefinite period at predetermined conditions. In certain instances, the airflow storage 216 is configured as a pressure vessel for maintaining a pressurized gas such as air.

The airflow storage 216 optionally may include valves, actuators, agitators, conduits, or additional devices to facilitate retaining air or regulating airflow. More specifically, airflow storage 216 may have a blow off or dump valve 218. The dump valve 218 may be configured to release air from the airflow storage to atmosphere (ATM) when a given property or parameter is met, for instance when pressure reaches a predetermined level. Alternatively, dump valve 218 may be configured to release air when the direction of airflow through the airflow storage 216 is reversed.

Configured according to the description hereinabove, the airflow subsystem 210 provides air from the airflow source 212 to the airflow storage 216 via the airflow control 214. In the embodiment of FIG. 1, the airflow subsystem 210 provides air and airflow properties for forced introduction to each of the patients breathing orifices via the distribution system 230.

According to various embodiments of the present disclosure, the distribution system 230 is configured to distribute airflow to each breathing orifice of a patient. As such, distribution system 230 may have an oral conduit O, a left nare conduit L, and a right nare conduit R. The oral conduit O, the left nare conduit L, and the right nare conduit R may have duplicate components. More specifically, the left nare conduit L may comprise a control valve 232, a dump valve 233, a sensor(s) 234, as well as connection port 235. By way of the connection port 235, the left nare conduit L may couple to a nare interface 236. The right nare conduit R may have a control valve 242, a dump valve 243, sensor(s) 244, as well as a connection port 245. By way of the connection port 245, the right nare conduit may couple to a right nare interface 246. Likewise, the oral conduit O may have a control valve 252, a dump valve 253, sensor(s) 254, as well as a connection port 255. By way of the port 255, the oral conduit may couple to an oral interface 256.

Each valve 232, 242, 252 may be configured to change airflow properties from the airflow storage 216 or airflow source 212 into each respective conduit L, R, O. Each valve 232, 242, 252 may be configured as a valve, an actuator, an orifice or other device configurable to stop, redirect, control, or otherwise regulate the airflow from airflow storage 216 through the respective conduit L, R, O of system 200. Each valve 232, 242, 252 may provide a uni-directional or bi-directional path for the airflow through each respective conduit L, R, O. In certain instances, the each valve 232, 242, 252 is positioned between the patient and the airflow subsystem 210 to control airflow.

Each dump valve 233, 243, 253 may be configured to change or control airflow properties from the airflow subsystem 210, or airflow from the patient. Each valve 233, 243, 253 may be configured as a valve, an actuator, an orifice, or other device configurable to vent or dump airflow from each respective conduit L, R, O to atmosphere. As illustrated, dump valves 233, 243, 253 are positioned between the control valves and respective sensors; however, in other cases the dump valves may be positioned at any suitable location within the conduits.

Each sensor 234, 244, 254 is configured to sense at least one property of the airflow through and/or regarding each respective conduit L, R, O. The property may be airflow velocity or rate, pressure, temperature, humidity, density, or any other property of a gaseous fluid flow, without limitation. The airflow may be flowing to the patient or from the patient, and each sensor 234, 244, 254 (if present) measures properties of that airflow. Each sensor 234, 244, 254 may be considered either a single device sensing a single airflow property, a single device sending a plurality of airflow properties, or a plurality of devices each sensing a single airflow property. Each sensor 234, 244, 254 may be positioned in fluid communication with the conduits L, R, O between the patient and the airflow subsystem 210. Additionally, each sensor 234, 244, 254, may be positioned in fluid communication with the conduits L, R, O between the ports 235, 245 255 and each of the valves 232, 242, 252.

The nare interfaces 236, 246 are configured, in some embodiments, for positioning on the external surface of the left nare and the right nare, respectively, of the patient. Nare interfaces 236, 246 may comprise a nasal mask, a nasal pillow, or any other device positionable in operational relationship with the nose, nostrils, or nares. Nare interfaces 236, 246 are at the distal ends of the conduits L, R, and serve to fluidly couple airflow from system 200 to a patient's nose and more specifically, a patient's nasopharynx. In certain instances, nare interfaces 236, 246 are removable and replaceable components.

Oral interface 256 is configured to interface with patient's mouth by any external or internal means. Oral interface 256 may comprise an oral mask, a bite valve, a tube, or any device configurable for fluid communication with a patient's mouth, and more specifically, a patient's nasopharynx. The oral interface 256 may be positioned at the distal ends of the oral conduit O, and serve to fluidly couple airflow to and/or from system 200 to a patient's mouth. In certain instances, the oral interface 256 is a removable and replaceable component.

Configured according to the positive pressure embodiments, the airflow distribution subsystem 230 provides air from the airflow subsystem 210, more specifically, from the airflow source 212 to some or all of the patient's breathing orifices. The airflow distribution subsystem 230 provides fluid communication for airflow's forced introduction to each of the patients breathing orifices by the oral conduit O, left nare conduit L, and right nare conduit R. The airflow subsystem 210 and the distribution subsystem 230 are at least partially controlled by the control subsystem 260.

According to various embodiments of the present disclosure, the control subsystem 260 includes the electric power supply 262, a display or visual interface 264, a memory or computer readable medium 266, and a processor 268. The control subsystem 260 is operatively coupled to substantially all components of the system 200, including: sensors 234, 244, 254; control valves 232, 242, 252; dump valves 233, 243, 253; pressure source 212; pressure control 214; and dump valve 218.

Electric power supply 262 may be any device configurable for receiving and distributing electric power through system 200 as described herein. Electric power supply 262 may have wires or cables for connecting to an electric outlet. Further, electric power supply may be battery, capacitor, or other electric charge storage device. Electric power supply 262 may have an AC (alternating current) to DC (direct current) converter.

Visual interface 264 may be any device configurable to display alpha-numeric or graphical data. For example, visual interface 264 may be a screen, monitor, liquid crystal display (LCD), or other display without limitation. Visual interface 264 may be configured for user inputs, such as a touch screen or a keyboard. Further, visual interface 264 may be configured to work in conjunction with auditory or audible signals.

Memory 266 may include any processor readable or read/writable memory. Non-limiting examples of suitable memory devices are random access memory (RAM) or read only memory (ROM). In alternate configurations the memory 226 may be a secure digital interface card, a universal serial bus (USB) memory, or other portable memory devices. Memory 266 may have RAM to provide a working area for a processor to temporarily store data and to execute programs. Memory 266 may have ROM to store the operating system, operating parameters, and user/operator-supplied programs to read data, record data, and produce data or indicia of a sensed property. Secure digital interface card may provide a portable aspect to memory 266, such that data may be transported from system 200 to another substantially similar system or to a computer. The memory 266 is thus an example of a non-transitory computer-readable medium.

Processor 268 may be any suitable computer processor configured to receive digital or analog signals from the system 200, access instructions on the memory 266, and provide and receive a signal from the visual interface 264. Additionally, the processor 268 provides commands to components of the system 200 to alter operation. In various embodiments, the processor 268 may be integral to the components of the control subsystem. Additionally, the processor 268 may be considered a controller or microcontroller.

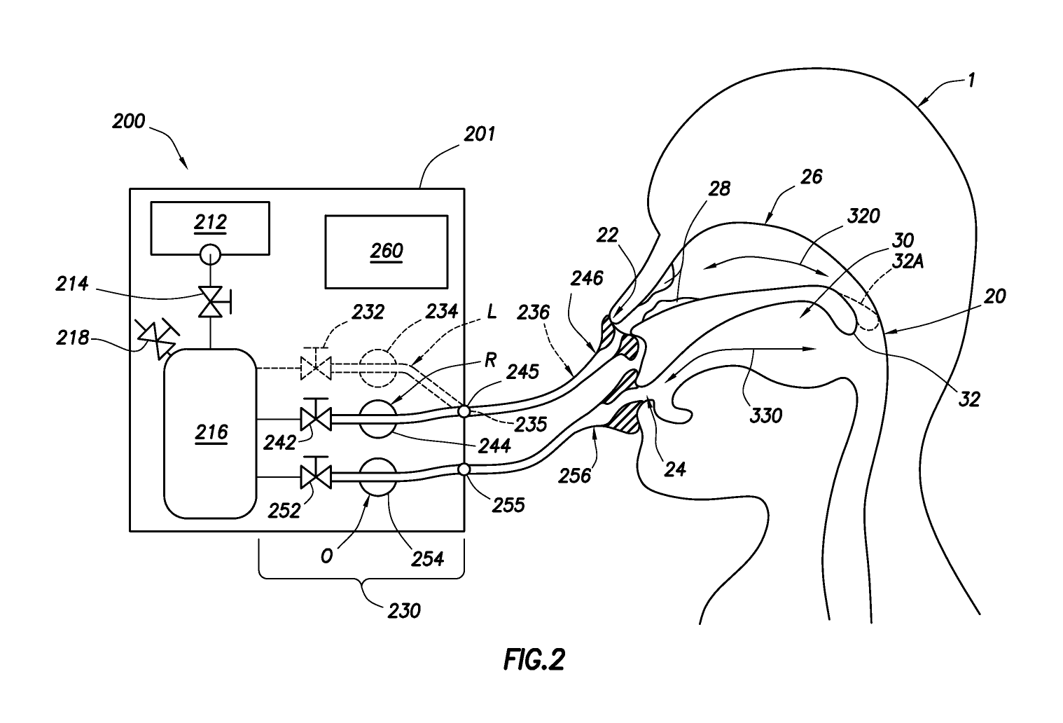

Referring now to FIG. 2, a simplified representation of system 200 is shown in fluid communication with the nasopharynx 20 of a patient 1. Patient 1 is illustrated in cross-section in FIG. 2, such that only one nare 22 is visibly in communication with system 200. As such, the arrangement and positioning of the nare conduits L, R may be interchangeable, though only the right nare conduit R is illustrated in FIG. 2 and described hereinbelow.

According to at least some embodiments, the distribution system 230 having oral conduit O, a left nare conduit L, and a right nare conduit R is in fluid communication with the nasopharynx 20. The nare conduits L, R of the distribution system 230 are in fluid communication with the nose, nasal openings, or nares 22 via the nasal interfaces 246, 236. Nasal interfaces 236, 246 couple to the system 200 by way of ports 245, 235 accessible through the cover 201. As illustrated, in some embodiments the nasal interface 246 is positioned adjacent the exterior of the nare 22 and forms a seal to prevent airflow leakage. The nasal interface 246 places the conduit R in fluid communication with nasopharynx 20 via nasal airflow 320 through nare 22, and nasal passage or nasal airway 26. Nasal airway 26 may have internal features 28 that provide resistance to nasal airflow 320. Internal features 28 may be the result of injury, disease, or other abnormal conditions without limitation. Additionally, the nasopharynx 20 includes the soft palate 32 that separates the oral airway 30 from the nasal airway 26.

The oral conduit O of the distribution system 230 is in fluid communication with the oral opening or mouth 24 via the oral interface 256. Oral interface 256 couples to the system 200 by way of port 255 accessible through cover 201. As illustrated, the oral interface 256 is positioned adjacent to the mouth 24 or partially in the mouth 24. When used, the oral interface 256 places the conduit O in fluid communication with nasopharynx 20 via oral airflow 330 through mouth 24 and oral airway 30.

According to various embodiments of the present disclosure, the system 200 is configured to measure nasal resistance in order to determine or diagnose the presence of the internal features 28 in the nasal airway 26 and/or attributes of the soft palate 32. Further, the system 200 is configured to determine the severity of the restriction in the nasal airway 26 created by the internal features 28. Additionally, the system 200 is configured to assist in measuring the resistance of the soft palate 32 to airflow in the nasal airway 26 and the oral airway 30.

The specification now turns to an explanation of a series of example method embodiments, which method embodiments use the positive pressure-based nasal function test device 200 described with respect to FIGS. 1 and 2. It is noted that not all of the components of the system 200 are needed in every example embodiment. Thus, a nasal function test device in accordance with FIGS. 1 and 2 shows many optional components, which need not be present in every case.

### Method Embodiment 1

FIG. 3 shows a method 300 in accordance with at least some embodiments. In particular, the method starts and proceeds to having the patient cease or pause breathing (block 302). The duration for the patient's pause in breathing may be relatively short (e.g., <1 minute) in order to ensure patient comfort. In a non-limiting example, the method 300 may proceed for between about 0.1 seconds (s) and about 0.3 s, and in one embodiment 0.2 s is used. Alternate periods may be sufficient for at least one determination of nasal resistance according to the current method 300. Longer durations for paused breathing or held breath may be utilized for multiple nasal resistance determinations according to method 300.

During the period of time when the patient has paused breathing, the method comprises forcing airflow into the left nare (block 304), and forcing airflow in the right nare (block 306). As illustrated, in some cases the forcing of airflow into the nares is performed simultaneously; however, in other cases the forcing of airflow into each nare may be performed consecutively by selective use of valves in the distribution subsystem 230 of the nasal function test device 200. Regardless of the order of the forcing of airflow into the nares, the pressure and flow is controlled such that a sufficient airflow provided to the nares escapes by way of the patient's mouth. That is, in some embodiments the airflow moves in the nares, and because the patient has ceased breathing, the airflow moves through the nasopharynx and out the mouth. In example cases, the positive pressure of air applied to obtain a sufficient airflow was between 4 and 6 centimeters of water (cmH2O), but other pressures may be used.

In this first example method, the indicia of nasal resistance is determined based on properties of the airflow into the nares, and no properties of the airflow out of the mouth are measured. Thus, portions of the illustrated distribution system 230 of FIGS. 1 and 2 directed the oral airway may be omitted in these cases. With airflow into the nares and out the mouth established, the nasal function test device measures and/or determines one or more properties of the airflow (block 308), and from those properties determines a value indicative of nasal resistance of one or both of the nares (block 310). Once the measurement of the properties of the airflow is complete (again block 308), the patient may be instructed to resume breathing.

Consider, as an example, that the sensors 234 and 244 each comprises an airflow sensor (that measures airflow velocity or rate), and a pressure sensor. Thus, in one example embodiment the nasal function test device measures an airflow into each nare, and a pressure of the airflow developed for each nare. From the pressure and airflow into each nare, a value indicative of nasal resistance may determined. Without limitation by any theory, determination of the nasal airflow resistance for each nare in these embodiments may be given by the Equation (1):

Rₙ(Resistance)=Pₙ(pressure,nare)/Fₙ(Flow,nare). (1)

wherein the nasal resistance for the particular nare is Rₙ, the pressure of the airflow forced into the nare is Pₙ, and the airflow rate into the nare is Fₙ. In some embodiments, particularly embodiments with lower cost nasal function devices, the nares may be treated as a singular entity, with airflow forced in the both nares by way of a common plenum, and the nasal resistance determined based on a single measured pressure and airflow; however, while the indicia of nasal resistance determined with nares considered a single entity may be useful, other information may be lost in such embodiments, such as differences in nasal resistance between the nares.

For situation where the nasal resistance of each nare is calculated, there may be instances where an indicia of total nasal resistance is desired. In such cases, an indicia of nasal resistance as calculated for each nare (such as Equation (1)) may be combined. Given that the nares are parallel pathways to the nasopharynx, the total nasal resistance may be determined by mathematically combining the individual resistances, such as the product of the resistance divided by the sum of the resistances.

Having both a pressure sensor and an airflow sensor for determining nasal resistance in these embodiments is merely illustrative. That is, while having both sensors may provide more accurate readings and therefore more accurate determinations of indicia of nasal resistance, in yet still other embodiments (particularly lower cost embodiments) the pressure sensor(s) may be omitted. That is, in embodiments where the pressure source 212 is an electric motor coupled to a blower, the motor speed and/or blower speed may be directly related to applied pressure. Thus, based on a predetermined table that relates motor/blower speed to applied pressure (e.g., stored in the memory 266 and accessible by the processor 268), the pressure portions of the measured data may be inferred by the processor from blower/motor speed as commanded or measured. Thus, operation of the example Equation (1) may be based on measured airflow, and a value for pressure inferred from the table based on blower/motor speed.

As implied by reference to FIGS. 1 and 2, the pressure measurements when pressure sensors are present are made within the nasal function test device 200, not directly within the nares of the patient. Thus, in some embodiments the pressure used in Equation (1) above may be adjusted to account for pressure drop encounter through the conduits between the pressure sensor and the entrance to the nare. That is, the airflow in these embodiments is measured, and knowing the size and length of the conduits between the pressure sensors and the patient's nares, the expected pressure drop can be calculated, and the adjusted pressure used in example Equation (1) above.

### Method Embodiment 2

Some patients may be unwilling or unable to temporarily pause breathing. Examples of patients unwilling or unable to pause breathing may comprise unconscious persons, infants, children, or just belligerent patients. In the second example embodiment, values indicative of nasal resistance may be determined from nasal-only determined properties in spite of the non-compliance regarding cessation of breathing. Still referring to FIG. 3, in these embodiments the illustrative method steps are substantially the same as the first example method embodiments, with the exception of the pause in breathing (block 302). That is, a value indicative of nasal resistance may still be determined in spite of the continued breathing of the patient. The applied pressure and airflow may be ramped until airflow is established out of the mouth, at least during the cross-over periods between inhalation and exhalation (or vice versa). In some cases, a positive pressure of 4 to 6 cmH2O may be applied, but other pressures are possible as well. Because of the respiration, airflow may not escape the mouth at all times—for example, during at least a portion of the inhalation, airflow may enter the mouth. However, establishing airflow into the nares and out of the mouth during at least one period of time ensures that the soft palate is in an open position or orientation.

The methodology to determine the value indicative of nasal resistance in spite of the breathing may take many forms. For example, the measurements of pressure and flow used to determine the value indicative of nasal resistance may be measured during periods of time between inhalation and exhalation (i.e., the slack time between breaths—either just before inhalation, or just before exhalation). In other cases, the measured values during an inhalation or during an exhalation are used, but the accuracy of the results may be skewed slightly because the patient's diaphragm may induce higher or lower pressure across the nasopharynx than would be present otherwise for a patient compliant with a requirement to cease breathing. In yet still further cases, some form of averaging between values measured during inhalation and the values measured during exhalation may be used. Nevertheless, the indicia of nasal resistance determined may still be useful in diagnosing various ailments in spite of the uncertainty introduced by the patient's respiration.

### Method Embodiment 3

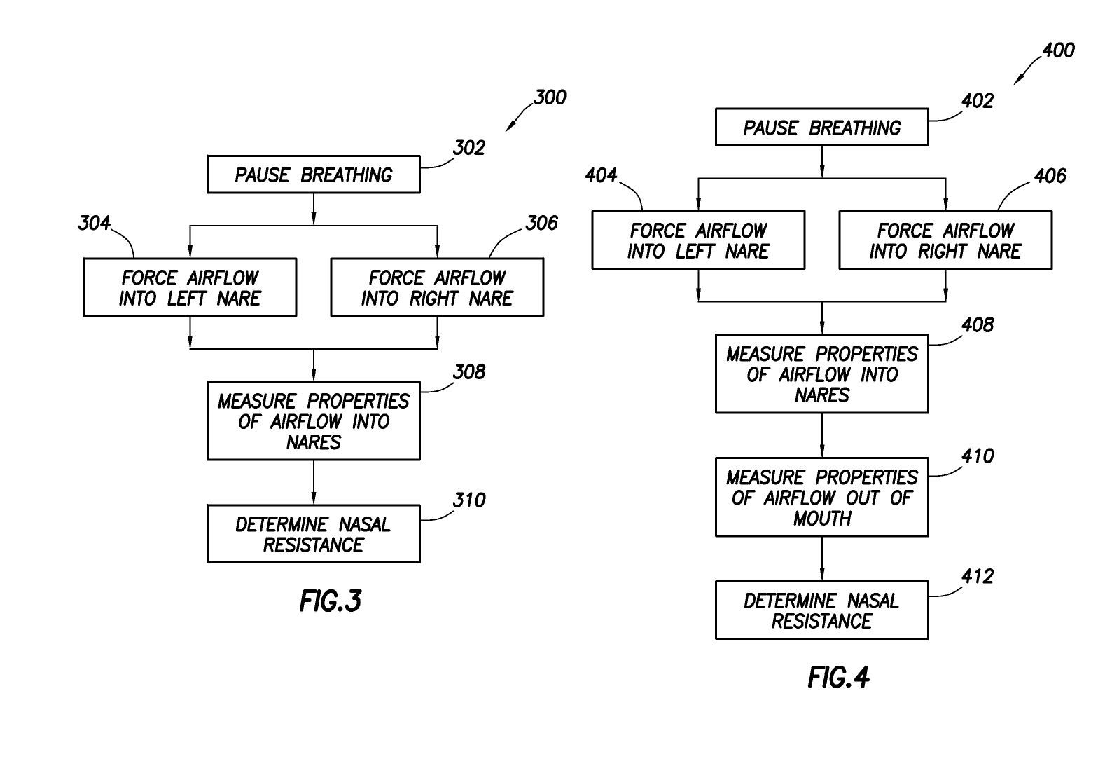

FIG. 4 shows a method in accordance with at least some embodiments. The illustrative method of FIG. 4 involves measuring parameters associated with airflow through the nares and the mouth in determining the values indicative of nasal resistance. In particular, the method starts and proceeds to having the patient cease or pause breathing (block 402). The duration for the patient's pause in breathing may be relatively short (e.g. <1 second) in order to ensure patient comfort. In a non-limiting example, the method 300 may proceed for between about 0.1 s and about 0.3 s, and in one embodiment 0.2 s is used. Alternate periods may be sufficient for at least one determination of nasal resistance according to the method 400. Longer durations for paused breathing or held breath may be utilized for multiple nasal resistance determinations according to method 400.

During the period of time when the patient has paused breathing, the method comprises forcing airflow into the left nare (block 404), and forcing airflow in the right nare (block 406). As illustrated, in some cases the forcing of airflow into the nares is performed simultaneously; however, in other cases the forcing of airflow into each nare may be performed consecutively by selective use of valves in the distribution subsystem 230 of the nasal function test device 200. Regardless of the order of the forcing of airflow into the nares, the pressure and flow is controlled such that a sufficient airflow provided to the nares escapes by way of the patient's mouth. That is, in some embodiments the airflow moves in the nares, and because of the positive pressure applied the airflow moves through the nasopharynx and out the mouth. In example cases, the positive pressure of air applied to obtain a sufficient airflow is between 4 and 6 cmH20, but other pressures may be used.

In this third example method, the indicia of nasal resistance is determined based on properties of the airflow into the nares, as well as properties of the airflow out of the mouth. With airflow into the nares and out the mouth established (blocks 404 and 406), the nasal function test device measures and/or determines one or more properties of the airflow into the nares (block 408), and one or more properties of airflow out of the mouth (block 410). From the properties determined, the method comprises determining a value indicative of nasal resistance of one or both of the nares (block 412). Once the measurement of the properties of the airflow is complete (again blocks 408 and 410), the patient may be instructed to resume breathing.

Consider, as an example, that the sensors 234 and 244 each comprise an airflow sensor (that measures airflow velocity or rate), and a pressure sensor. Moreover, consider that the sensor 254 associated with the oral airway comprises a pressure sensor. Thus, in one example embodiment the nasal function test device measures airflow into each nare, a pressure of the airflow developed for each nare, and the pressure associated with airflow out of the mouth. From the pressures and airflow, a value indicative of nasal resistance may be determined. Without limitation by any theory, determination of the values indicative of nasal resistance for each nare in these embodiments may be given by the Equation (2):

Rₙ(Resistance)=[Pₙ(pressure,nare)−Pₘ(pressure mouth)]/Fₙ(Flow,nare). (2)

wherein nasal resistance is Rₙ, the pressure measured associated with the mouth is Pₘ, the pressure of the airflow forced into the nare is Pₙ, and the airflow rate into the nare is Fₙ. In some embodiments, particularly embodiments with lower cost nasal function devices, the nares may be treated as a singular entity, with airflow forced in the both nares by way of a common plenum, and the nasal resistance determined based on a single measured pressure and airflow with respect to the nares; however, while the indicia of nasal resistance determined with nares considered a single entity may be useful, other information may be lost in such embodiments, such as differences in nasal resistance between the nares. Further, the indicia of nasal resistance of each nare can be combined into an overall indicia of nasal resistance in any suitable mathematical way.

Having a both a pressure sensor and an airflow sensor for each nare in these embodiments is merely illustrative. That is, while having both sensors may provide more accurate readings and therefore more accurate determinations of indicia of nasal resistance, in yet still other embodiments (particularly lower cost embodiments), the pressure sensors for the nares may be omitted, and the nasal function test device may have only a single pressure sensor arranged to measure the pressure associated with the mouth. That is, in embodiments where the pressure source 212 is an electric motor coupled to a blower, the motor speed and/or blower speed may be directly related to applied pressure. Thus, based on a predetermined table that relates motor/blower speed to applied pressure (e.g., stored in the memory 266 and accessible by the processor 268), the pressure portions of the measured data may be inferred by the processor from blower/motor speed as commanded or measured. Thus, operation of the example Equation (1) may be based on measured airflow, and a value for pressure at the nares inferred from the table based on blower/motor speed.

In yet still further embodiments, the indicia of nasal resistance may use just the measured pressure associated with the mouth in the determination. That is, the indicia of nasal resistance in these embodiments may be calculated using Equation (1) above, where the pressure value used is the pressure measurement associated with the mouth rather than the applied pressure at the nares. Here again, the pressure measurements are made within the nasal function test device 200, not directly at within the nares or mouth of the patient. Thus, in some embodiments the pressures used in Equation (2) above may be adjusted to account for pressure drop encounter through the conduits between the pressure sensor and the patient. That is, the airflow in these embodiments may be measured, and knowing the size and length of the conduits between the pressure sensors and the patient, the expected pressure drop can be calculated, and the adjusted pressures used in example Equation (2) above.

In the embodiments discussed with respect to method embodiment 3, at least a portion of the airflow from the mouth is directed through the nasal function test device 200, but no airflow is provided to the mouth. Thus, in these embodiments the oral conduit within the nasal function test device may be fluidly coupled to the sensor 254 and valve 252, but a fluid connection to the airflow subsystem 210 may be omitted, or the control valve 252 closed and dump valve 253 opened. In some further cases, the sensor 254 may be only a pressure sensor for the example method embodiment 3—for example where the pressure measured is not compensated for distance and airflow between the mouth and the pressure sensor within the nasal function test device.

### Method Embodiment 4

Again, some patients may be unwilling or unable to temporarily pause breathing. Still referring to FIG. 4, in these embodiments the illustrative method steps are substantially the same as the third example method embodiments, with the exception of the pause in breathing (block 402). That is, a value indicative of nasal resistance may still be determined in spite of the continued breathing of the patient. The applied pressure and airflow may be ramped until airflow is established out of the mouth, at least during the respiration slack times. In some cases, a positive pressure of 4 to 6 cmH20 may be applied, but other pressures are possible as well. Because of the respiration, airflow may not escape the mouth at all times—for example, during at least a portion of the inhalation airflow may enter the mouth. However, establishing airflow into the nares and out of the mouth during at least one period of time ensures that the soft palate is in an open position.

The methodology to determine the value indicative of nasal resistance in spite of the breathing may take many forms. For example, the measurements of pressure and airflow used to determine the value indicative of nasal resistance may be measured during periods of time between inhalation and exhalation (i.e., the slack time between breaths, either just before inhalation, or just before exhalation). In other cases, the measured values used are measured during an inhalation or during an exhalation. In these embodiments, the measurement of the pressure of airflow associated with the mouth may compensate, at least in part, for the patient respiration.

Consider, as an example, measurements taken during periods of time when the patient is inhaling. During an inhalation, the diaphragm retracts, drawing air into the lungs. Thus, when the diaphragm retracts the pressure in the lungs, as well as within the nasopharynx, is reduced. Stated otherwise, considered with respect to the case where the patient has paused breathing, for the same airflow forced into the nares, the pressure associated with the mouth may be lower. By measuring the pressure associated with the mouth, the pressure drop is known or substantially known, in spite of the contribution in pressure drop caused by the diaphragm. Thus, though inaccuracies may be introduced by the inhalation of the patient, the inaccuracies may be reduced or eliminated by the measure of pressure associated with the mouth. The opposite situation (i.e., when the patient is exhaling) may also be addressed in a similar manner.

In yet still further cases, some form of averaging between values measured during inhalation and the values measured during exhalation may be used. Nevertheless, the values indicative of nasal resistance determined may still be useful in diagnosing various ailments in spite of the uncertainty introduced by the patient's respiration.

### Method Embodiment 5

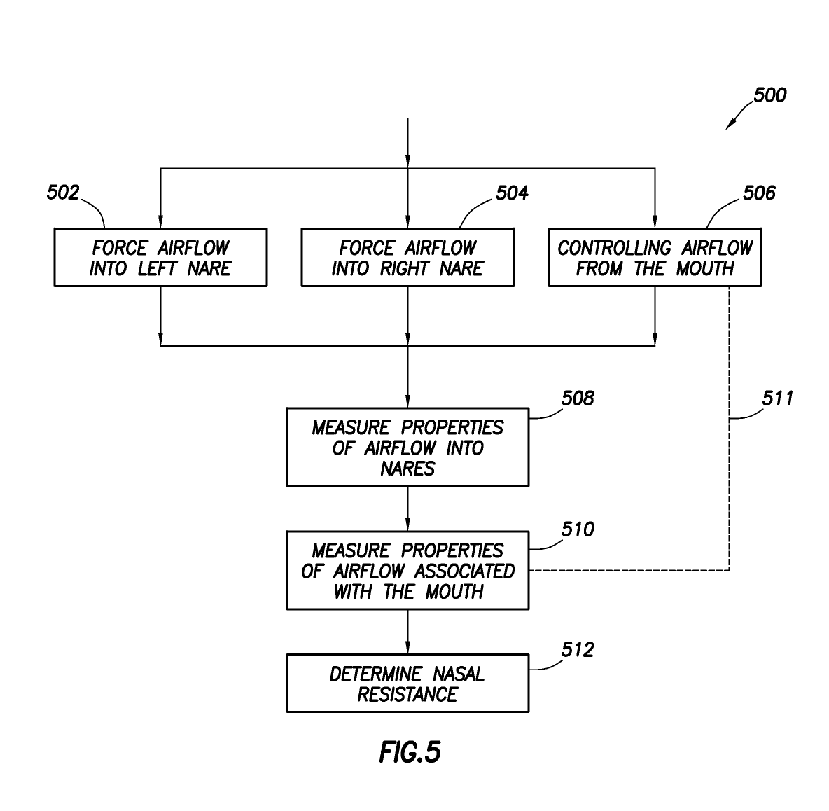

FIG. 5 shows a method 500 in accordance with at least some embodiments. The illustrative method of FIG. 5 involves, during patient respiration, measuring parameters associated with airflow through the nares and mouth, and determining the indicia of nasal resistance. In particular, during example embodiment 5 the patient may be breathing normally, or may be instructed to breathe in some particular fashion (e.g., slow and shallow breaths, slow breaths to tidal volume, quick and shallow breaths, quick breaths to tidal volume).

During periods of time when the patient is breathing, the method comprises forcing airflow into the left nare (block 502), and forcing airflow into the right nare (block 504). As illustrated, in some cases the forcing of airflow into the nares is performed simultaneously; however, in other cases the forcing of airflow into each nare may be performed consecutively by selective use of valves in the distribution subsystem 230 of the nasal function test device 200. Regardless of the order of the forcing of airflow into the nares, in some cases the pressure and flow provided the nares is controlled such that a sufficient airflow provided to the nares escapes by way of the patient's mouth, at least during the respiration slack times. In some cases, a positive pressure of 4 to 6 cmH20 may be applied, but other pressures are possible as well. Because of the respiration, airflow may not escape the mouth at all times. However, establishing airflow into the nares and out of the mouth during at least one period of time ensures that the soft palate is in an open position.

The illustrative method further comprises controlling airflow with respect to the patient's mouth (block 506). The controlling may take many forms. In one example variation the pressure associated with the airflow of the patient's mouth may be measured by the sensor 254 in the nasal function test device 200, and the valves 252 and/or 253 adjusted to control pressure to be about constant. The control may require limiting the escaping airflow, providing positive pressure from the airflow subsystem 210, or both depending on the respiratory state (i.e., inhalation or exhalation), and the rate of airflow escaping out of the mouth. In yet still other cases, the airflow into the nares may be adjusted to control the pressure in the mouth to be about constant.

In this fifth example method, the values indicative of nasal resistance is determined based on properties of the airflow into the nares, as well as properties of the airflow associated with the mouth, which again may be controlled. With airflow into the nares (again blocks 502 and 504), and controlled airflow with the respect to the mouth (again block 506), the nasal function test device measures and/or determines one or more properties of the airflow into the nares (block 508), and one or more properties of airflow associated with the mouth (block 510). Dashed line 511 indicates that the control illustrated by block 506 may be based on the same properties measured, as indicated by block 510. From the properties a value indicative of nasal resistance of one or both of the nares is determined (block 512).

Consider, as an example, that the sensors 234 and 244 each comprises an airflow sensor (that measures airflow velocity or rate), and a pressure sensor. Moreover, consider that the sensor 254 associated with the oral airway comprises a pressure sensor. Thus, in one example embodiment the nasal function test device measures airflow into each nare, a pressure of the airflow developed for each nare, and the airflow pressure associated the mouth (and controls based on the measured pressure). From the pressures and airflow, a value indicative of nasal resistance may determined, such as by use of Equation (2) above. In some embodiments, particularly embodiments with lower cost nasal function devices, the nares may be treated as a singular entity, with airflow forced in the both nares by way of a common plenum, and the nasal resistance determined based on a single measured pressure and airflow with respect to the nares; however, while the indicia of nasal resistance determined with nares considered a single entity may be useful, other information may be lost in such embodiments, such as differences in nasal resistance between the nares.

Having both a pressure sensor and an airflow sensor for each nare in these embodiments is merely illustrative. That is, while having both sensors may provide more accurate readings and therefore more accurate determinations of indicia of nasal resistance, in yet still other embodiments (particularly lower cost embodiments), the pressure sensor(s) for the nares may be omitted, and the nasal function test device may have only a single pressure sensor arranged to measure the pressure associated with the mouth. That is, in embodiments where the pressure source 212 is an electric motor coupled to a blower, the motor speed and/or blower speed may be directly related to applied pressure. Thus, based on a predetermined table that relates motor/blower speed to applied pressure (e.g., stored in the memory 266 and accessible by the processor 268), the pressure portions of the measured data may be inferred by the processor from blower/motor speed as commanded or measured. Thus, operation of the example Equation (s) may be based on measured airflow, and a value for pressure inferred from the table based on blower/motor speed.

In yet still further embodiments, determining the values indicative of nasal resistance may use just the measured pressure associated with the mouth. That is, the values indicative of nasal resistance in these embodiments may be calculated using Equation (1) above, where the pressure value used is the pressure measurement associated with the mouth rather than the applied pressure at the nares. Here again, and regardless of the embodiment, the pressure measurements when pressure sensors are present are made within the nasal function test device 200, not directly at within the nares or mouth of the patient. Thus, in some embodiments the pressures used may be adjusted to account for pressure drop encounter through the conduits between the pressure sensor and the patient. That is, the airflow in these embodiments may be measured, and knowing the size and length of the conduits between the pressure sensors and the patient, the expected pressure drop can be calculated, and the adjusted pressures used.

In yet still further example embodiments, property measured with respect to the mouth may be airflow rather than pressure, and thus the sensor 254 may be an airflow sensor. That is, the indicia of nasal resistance in these embodiments may be calculated using Equation (1) above, where the pressure value used is the pressure measurement used is the applied pressure at the nares; however, controlling airflow associated with the mouth, the pressure within the mouth may effectively be controlled in spite not directly measuring the pressure.

### Method Embodiment 6

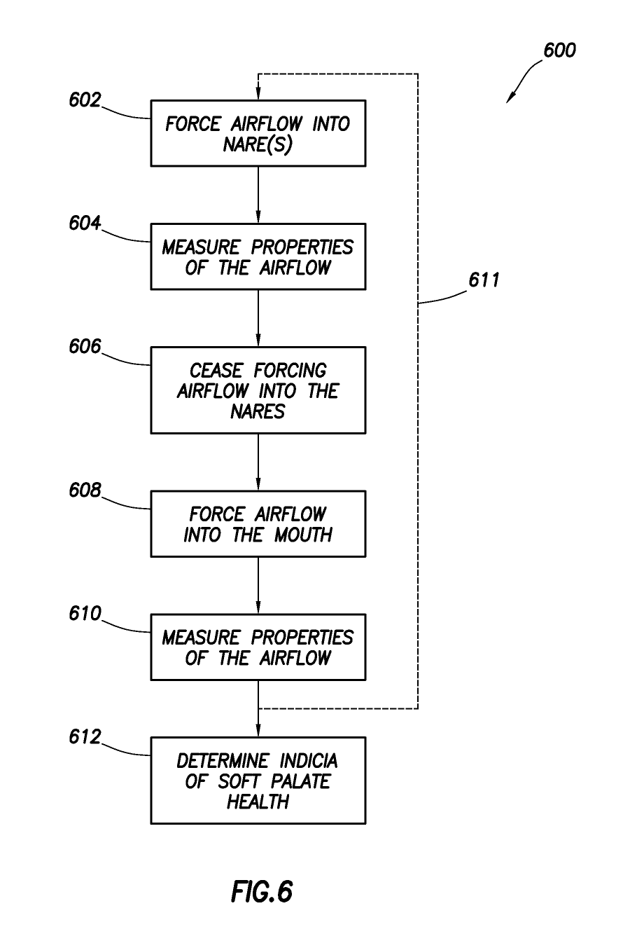

FIG. 6 shows a method 600 in accordance with at least some embodiments. The illustrative method of FIG. 6 involves determining values indicative of nasal resistance, but in this case associated with the health of the soft palate 32 of the patient 1. In particular, method 600 involves forcing airflow into one or both nares (block 602), and measuring a property of the airflow (block 604). In some cases, the airflow is established before the measurement takes places, and in other cases the establishing and measuring may occur simultaneously. Regardless of the precise time, the time that airflow is provided is sufficient to move or place the soft palate 32 in an open orientation (shown in solid lines in FIG. 2). Regardless of the precise predetermined period of time (e.g., up to 1 s in some cases, and up to 5 s in other cases), the forcing of airflow eventually ceases (block 606), and airflow is forced in the opposite or reverse direction.

In particular, the method then comprises forcing airflow into the mouth (block 608) with the goal of forcing the soft palate to close, and measuring a property of the airflow (block 610). A closed soft palate is shown in dashed lines in FIG. 2 as soft palate 32A. In some cases, the airflow is established before the measuring takes place, and in other cases the measurement may occur simultaneously. Before proceeding, a few points are in order. First, airflow into the mouth will flow past the soft palate and out the nares only momentarily, and such flow tends to cause the soft palate to close or block the nasopharynx. Thus, in most patients the reverse flow that exits the nares may be very small, or even nonexistent. A small percentage of patients may be able to sustain the reverse airflow, and this ability too may be indicative of health of the soft palate. In many cases, the forcing of airflow repeats at least once, as illustrated by line 611.

The method proceeds to determining an indicia of soft palate health (block 612), which is a component of the overall nasal resistance. The indicia of soft palate heath may take many forms. In some cases, the rise time to establish flow from the nares through mouth after the soft palate has closed (i.e., just after a “reverse” flow”), may be an indication of soft palate health, with rise time indicative of how quickly the soft palate may move to an open orientation. Likewise, the amount of reverse flow before the soft palate closes (i.e., just after a forward flow), and/or the time it takes the soft palate to close after application of a known volume or rate of airflow, may be an indication of soft palate health.

In yet still further cases, a measure of flexibility of the soft palate may be determined as indication of soft palate health, where the measure of flexibility is determined by alternating the forward and reverse flow with volumes and/or rates in each state just sufficient to open and close, respectively, the soft palate, and where the frequency at which such oscillations of the soft palate can be induced is an indication of the soft palate health.

In many embodiments, the nasal function test device 200 may be coupled to the patient for a very short period of time, for example the amount of time used for a single nasal resistance determination for each nare. However, in other cases the nasal function test device may be coupled to the patient for an extended period of time, for example over night. In such cases, the time varying nature of the patient's nasal resistance may be determined. However, in embodiments where the patient may be coupled to the device for an extended period, the possibility exists that expelled carbon dioxide may build up in the conduits in such a way as to limit the oxygen flowing to the patient. To address such situations, in some embodiments the nasal interfaces and/or oral interface may have an aperture representing a controlled leak for the escape of carbon dioxide. In other cases, the dump valves 233, 243, 253 may be fully or partially opened to enable exhaled carbon dioxide to escape the system. In either case, the controlled leak will represent a known or pre-determined leak, and as such may be compensated for in the determinations of nasal resistance.

The various embodiments discussed to this point have been based on devices that apply positive pressure to one or more breathing orifices of the patient. The positive pressure applied induces airflow through the nose and mouth of the patient, and a value indicative of nasal resistance is determined. However, in other embodiments, the same or similar tests may be performed by applying negative pressure (i.e., vacuum) to one or more breathing orifices to establish airflow. Again, a value indicative of nasal resistance may be determined based on the airflow. More specifically, in some cases a negative pressure is applied to the patient's mouth, which induces airflow into the nares and out the mouth. In other cases, negative pressure is applied to one or both of the patient's nares, thereby inducing airflow into the patient's mouth. The airflow may have a measureable property, such as velocity (i.e. rate), pressure, or volume. Various properties of the airflow associated with the nares or the mouth may be measured. The measured properties are then compared and analyzed by the processor according to instructions stored on a memory device.

Some embodiments of the method also include adjusting the induced airflow in response to the measurements taken at the mouth and/or nares. More specifically, the measurements taken at the mouth or nare may serve to coordinate certain properties of the introduced airflow with the patient's respiration. The measurements related to the mouth and each nare are compared by the processor and adjustments are made according to instructions stored on the memory device.

Negative Pressure System:

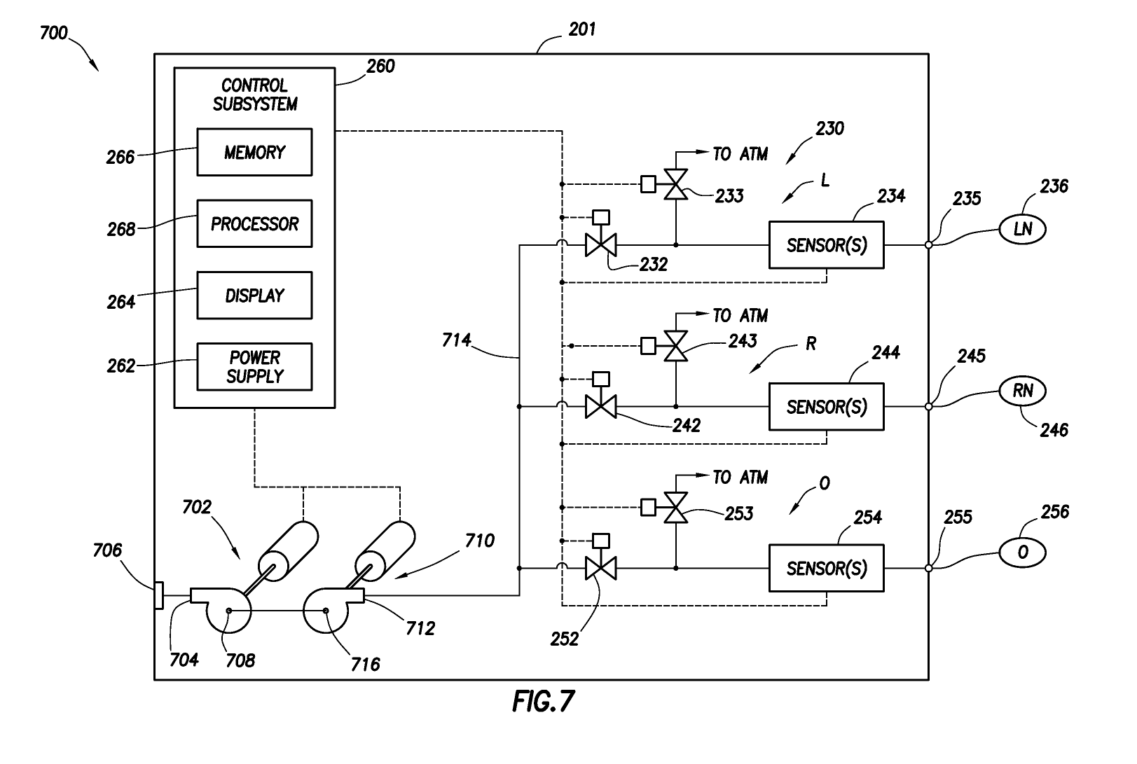

FIG. 7 illustrates a system 700 that operates, at least in part, on application of negative pressure to one or more of the patient's breathing orifices. Much like system 200, the system 700 comprises both electrical connections and fluid connections, and for purposes of clarity electrical connections will be shown with dashed lines, and fluid connections with solid lines. Moreover, many components implemented in the positive pressure system 200 can be implemented in a negative pressure system 700, and thus operation of devices introduced with respect to the system 200 that likewise may be present in the system 700 carry the same reference numbers, and will not be re-introduced.

Illustrative system 700 comprises a first blower and motor combination 702 (hereafter just blower 702). The blower 702 defines an outlet 704 fluidly coupled to a filter 706. Moreover, the blower 702 defines an inlet 708. When a fan or rotor of the blower 702 turns in a particular direction, airflow is drawn in the inlet 708 and directed out the outlet 704. The motor of the blower 702 may be an alternating current (AC) motor, a direct current (DC) motor, or a stepper motor. As illustrated, the motor of the blower may electrically and thus operatively couple to the control subsystem 260. The system 700 further comprises a second blower and motor combination 710 (hereafter just blower 710). The blower 710 defines an outlet 712 fluidly coupled to a header 714, which header 714 couples to the oral conduit O, the left nare conduit L, and the right nare conduit R. Moreover, the blower 710 defines an inlet 716 fluidly coupled to the inlet 708 of the blower 702. The motor of the blower 710 may be an alternating current (AC) motor, a direct current (DC) motor, or a stepper motor. As illustrated, the motor of the blower 710 may electrically and thus operatively couple to the control subsystem 260.

Although the system 700 is described as a “negative pressure” system, the presence and arrangement of blowers 702 and 710 enables the system 700 to selectively apply either positive pressure to the header 714 (and thus to any of the conduits O, L, and/or R) or to apply negative pressure to the header 714. That is, in operational situations where positive pressure is to be applied to one or more of the conduits (and thus one or more of the breathing orifices), blower 710 may be activated by the control subsystem 260 such that airflow is drawn through the filter 706 and blower 702, through the blower 710, thus applying positive pressure to the header 714. In situations where positive pressure is applied to the header 714, the blower 702 may be turned off or the motor slowed such that blower 710 overcomes any pressure developed by the blower 702. Thus, system 700 may be used to implement any of the illustrative methods discussed above, and thus calculate any of the illustrative values indicative of nasal resistance.

Applying negative pressure to the header 714 (and thus the conduits and respective breathing orifices) works opposite the positive pressure implementation. That is, in operational situations where negative pressure is to be applied to one or more of the conduits (and thus one or more of the breathing orifices), blower 702 may be activated by the control subsystem 260 such that airflow is drawn through blower 710, through the blower 702, and out the filter 706. In situations where negative pressure is applied to the header 714, the blower 710 may be turned off or the motor slowed such that blower 702 overcomes any pressure developed by the blower 710.

Discussing conduit L as representative of all the conduits, in situations where positive pressure is applied to the header 714, positive pressure may be applied to the conduit L and thus airflow forced through the conduit to the left nare. In situations where negative pressure is applied to the header 714, negative pressure may be applied to the conduit L and thus airflow drawn from the left nare through the conduit. Regardless of the pressure on the header 714, valve 232 may be closed and valve 233 opened thus enabling airflow either through the valve 233 and sensor 234 to the left nare, or airflow from the left nare through the sensor 234 and out the valve 233. By operating respective valves, the conduit R and conduit O may likewise take the various operational states.

The ability to apply a negative pressure to the conduits enables the ability to perform a variety of complementary nasal function tests to those tests discussed with respect to the positive pressure system 200. Thus, the specification now turns to an explanation of a series of example method embodiments, which method embodiments may use system 700. It is noted that not all of the components of the system 700 are needed in every example embodiment. Thus, a nasal function test device in accordance with FIG. 7 shows many optional components, which need not be present in every case.

### Method Embodiment 7

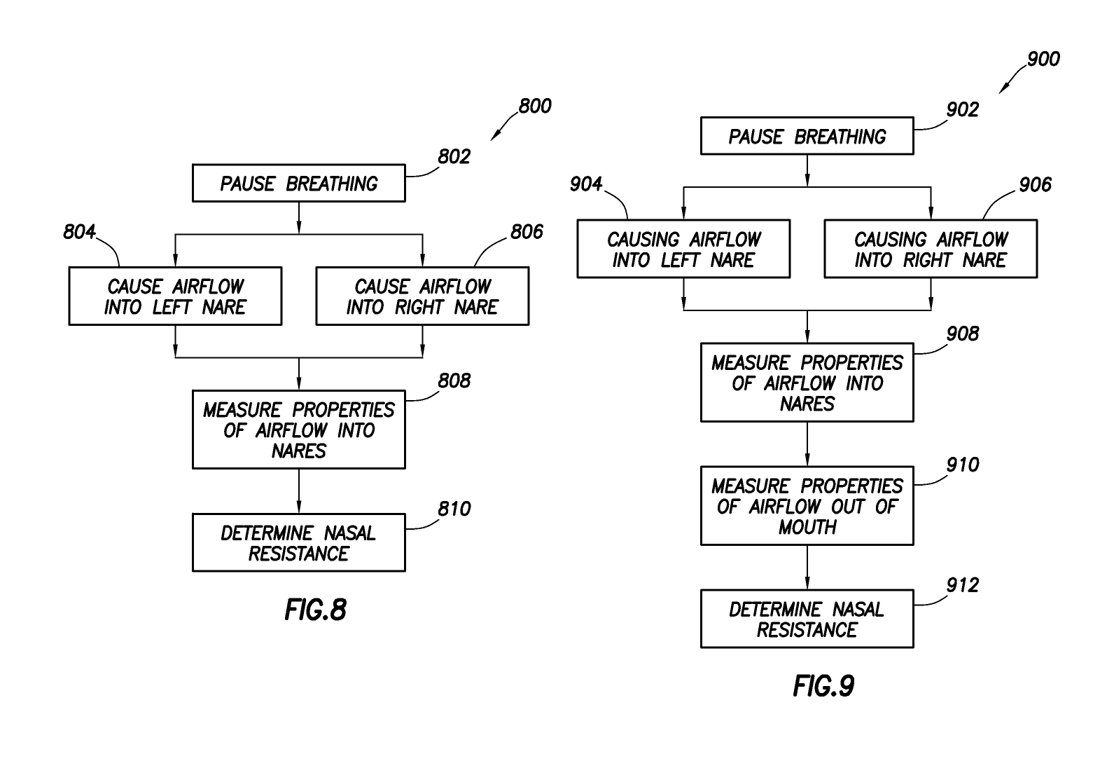

FIG. 8 shows a method 800 in accordance with at least some embodiments. In particular, the method starts and proceeds to having the patient cease or pause breathing (block 802). The duration for the patient's pause in breathing may be relatively short (e.g., <1 minute) in order to ensure patient comfort. In a non-limiting example, the method 800 may proceed for between about 0.1 s and about 0.3 s, and in one embodiment 0.2 s is used. Alternate periods may be sufficient for at least one determination of nasal resistance according to the current method 800. Longer durations for paused breathing or held breath may be utilized for multiple nasal resistance determinations according to method 800.

During the period of time when the patient has paused breathing, the method comprises causing airflow into the left nare (block 804), and causing airflow in the right nare (block 806). In the positive pressure embodiments, the causing of airflow is by applying positive pressure to the nares; however, the in negative pressure embodiments the causing of airflow is by applying a negative pressure to the patient's mouth, which thus draws airflow into the nares, around the soft palette, and out the mouth. In some cases the causing of airflow into the nares is performed simultaneously; however, in other cases the causing of airflow into each nare may be performed consecutively by selective use of valves in the distribution subsystem 230 of the nasal function test device 700. In example cases, the negative pressure applied to the mouth may be between 1 and 12 cmH20 below atmospheric, and more particularly between 6 and 8 cmH2O below atmospheric, but other negative pressures may be used.

In this seventh example method, the values indicative of nasal resistance may determined based on properties of the airflow into the nares. With airflow into the nares and out the mouth established based on negative pressure applied to the mouth, the nasal function test device measures and/or determines one or more properties of the airflow (block 808), and from those properties determines a value indicative of nasal resistance of one or both of the nares (block 810). Once the measurement of the properties of the airflow is complete (again block 808), the patient may be instructed to resume breathing.

Consider, as an example, that the sensors 234 and 244 each comprises an airflow sensor (that measures airflow velocity or rate), and a pressure sensor. Thus, in one example embodiment the nasal function test device measures an airflow into each nare, and a pressure of the airflow developed for each nare. From the pressure and airflow into each nare, a value indicative of nasal resistance may determined. Without limitation by any theory, determination of the value indicative nasal resistance for each nare in these embodiments may be given by the Equation (3):

Rₙ(Resistance)=Pₙ(pressure,nare)/Fₙ(Flow,nare). (3)

wherein the nasal resistance for the particular nare is Rₙ, the absolute value of the pressure of the airflow forced into the nare is Pₙ, and the airflow rate into the nare is Fₙ. In some embodiments, particularly embodiments with lower cost nasal function devices, the nares may be treated as a singular entity, with airflow drawn in the nares by way of a common plenum, and the value indicative of nasal resistance determined based on a single measured pressure and airflow; however, while the value indicative of nasal resistance determined with nares considered a single entity may be useful, other information may be lost in such embodiments, such as differences in nasal resistance between the nares.

For situation where the nasal resistance of each nare is calculated, there may be instances where an indicia of total nasal resistance is desired. In such cases, a value indicative of nasal resistance as calculated for each nare (such as Equation (3)) may be combined. Given that the nares are parallel pathways to the nasopharynx, the total nasal resistance may be determined by mathematically combining the individual resistances, such as the product of the resistance divided by the sum of the resistances.

As before, for patients that cannot or will not temporarily cease breathing, the nasal function test device of system 700 may be used on a patient that is breathing by either performing the nasal function test during slack times of breathing, by averaging results taken during inhalation and exhalation, or both.

### Method Embodiment 8

FIG. 9 shows a method 900 in accordance with at least some embodiments. The illustrative method of FIG. 9 involves measuring parameters associated with airflow through the nares and the mouth in the determining the values indicative of nasal resistance. In particular, the method starts and proceeds to having the patient cease or pause breathing (block 902).

During the period of time when the patient has paused breathing, the method comprises causing airflow into the left nare (block 904), and causing airflow in the right nare (block 906). In the pressure embodiments, the causing of airflow is by applying positive pressure to the nares; however, in the negative pressure embodiments the causing of airflow is by applying a negative pressure to the patient's mouth, which thus draws airflow into the nares, around the soft palette, and out the mouth. In some cases the causing of airflow into the nares is performed simultaneously; however, in other cases the causing of airflow into each nare may be performed consecutively by selective use of valves in the distribution subsystem 230 of the nasal function test device 700. In example cases, the negative pressure applied to the mouth may be between 1 and 12 cmH20 below atmospheric, and more particularly between 6 and 8 cmH2O below atmospheric, but other negative pressures may be used.

In this eighth example method, the value indicative of nasal resistance is determined based on properties of the airflow into the nares, as well as properties of the airflow out of the mouth. With airflow into the nares and out the mouth established (blocks 904 and 906), the nasal function test device measures and/or determines one or more properties of the airflow into the nares (block 908), and one or more properties of airflow out of the mouth (block 910). From the properties determined, the method comprises determining a value indicative of nasal resistance of one or both of the nares (block 912). Once the measurement of the properties of the airflow is complete (again blocks 908 and 910), the patient may be instructed to resume breathing.

Consider, as an example, that the sensors 234 and 244 each comprise an airflow sensor (that measures airflow velocity or rate), and consider that the sensor 254 associated with the oral airway comprises a pressure sensor. Thus, in one example embodiment the nasal function test device measures airflow into each nare, and the pressure associated with airflow out of the mouth. From the pressures and airflow, a value indicative of nasal resistance may be determined using Equation (3) above, where the pressure Pₙ is the absolute value of the pressure associated with the patient's mouth.

Now consider, as an example, that the sensors 234, 244, and 254 are all airflow sensors. In these embodiments, the value indicative of nasal resistance for each nare may be based on the relationship of the airflow into the particular nare to the total airflow (i.e., the airflow out the patient's mouth). The values indicative of nasal resistance in these cases may be particularly suited for determining imbalances in airflow between the nares. As a somewhat related example, consider that the sensors 234, 244, and 254 are all pressure sensors. In these embodiments, the value indicative of nasal resistance for each nare may be based on the relationship of the pressure associated with each nare to the pressure associated with the mouth. Here again, the values indicative of nasal resistance in these cases may be particularly suited for determining imbalances as between the nares.

Now consider, as yet another example, that the sensors 234 and 244 each comprise an airflow sensor, and a pressure sensor. Moreover, consider that the sensor 254 associated with the oral airway comprises a pressure sensor. Thus, in one example embodiment the nasal function test device measures airflow into each nare, a pressure of the airflow developed for each nare, and the pressure associated with airflow out of the mouth. From the pressures and airflows, a value indicative of nasal resistance may be determined. Without limitation by any theory, determination of the value indicative of nasal resistance for each nare in these embodiments may be given by the Equation (4):

Rₙ(Resistance)=[Pₘ(pressure mouth)−Pₙ(pressure,nare)]/Fₙ(Flow,nare). (4)

wherein nasal resistance is Rₙ, the negative pressure measured associated with the mouth is Pₘ, the negative pressure of the drawn through each nare is Pₙ, and the airflow rate into the nare is Fₙ. In some embodiments, particularly embodiments with lower cost nasal function devices, the nares may be treated as a singular entity, with airflow forced in the both nares by way of a common plenum, and the nasal resistance determined based on a single measured pressure and airflow with respect to the nares; however, while the value indicative of nasal resistance determined with nares considered a single entity may be useful, other information may be lost in such embodiments, such as differences in nasal resistance between the nares. Further, the value indicative of nasal resistance of each nare can be combined in any suitable mathematical way.

Having a both a pressure sensor and an airflow sensor for each nare in these embodiments is merely illustrative. That is, while having both sensors may provide more accurate readings and therefore more accurate determinations of values indicative of nasal resistance, in yet still other embodiments (particularly lower cost embodiments), the pressure sensor associated with the mouth may be omitted, and the nasal function test device may have pressure sensors associated only with the nares. That is, using blower 704, the motor speed and/or blower speed may be directly related to applied pressure. Thus, based on a predetermined table that relates motor/blower speed to applied pressure (e.g., stored in the memory 266 and accessible by the processor 268), the negative pressure applied to the mouth may be inferred by the processor from blower/motor speed as commanded or measured. Thus, operation of the example Equation (4) may be based on measured airflow and measured pressure at the nares, and a value for pressure at the mouth inferred from the table based on blower/motor speed.

As before, for patients that cannot or will not temporarily cease breathing, the nasal function test device of system 700 may be used on a patient that is breathing by either performing the nasal function test during slack times of breathing, by averaging results taken during inhalation and exhalation, or both.

### Method Embodiment 9

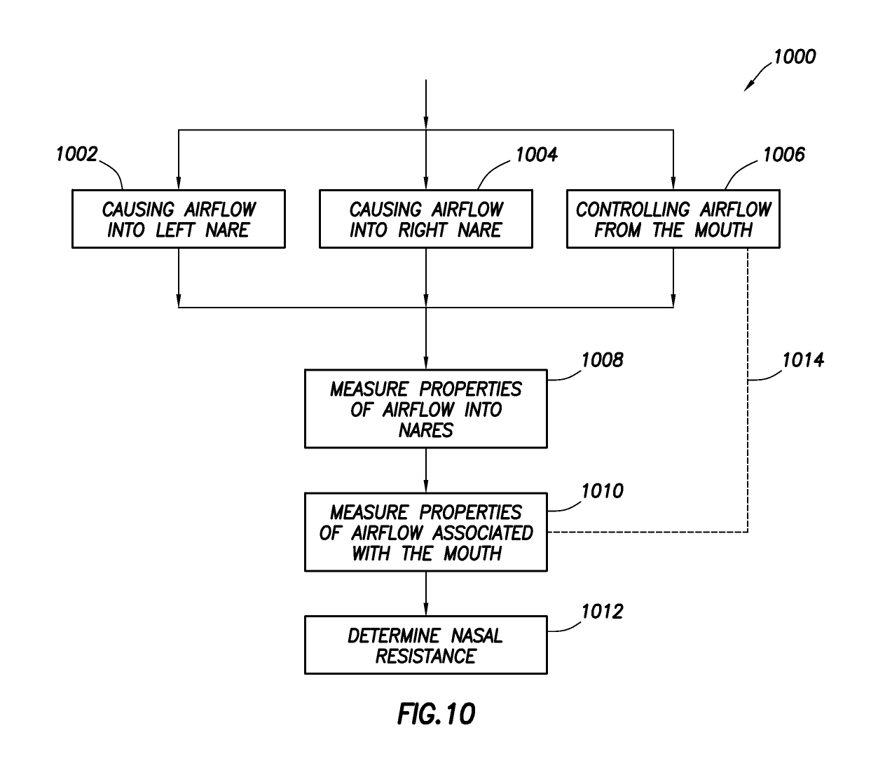

FIG. 10 shows a method 1000 in accordance with at least some embodiments. The illustrative method of FIG. 10 involves, during patient respiration, measuring parameters associated with airflow through the nares and mouth, and determining the values indicative of nasal resistance. In particular, during example embodiment 9 the patient may be breathing normally, or may be instructed to breathe in some particular fashion (e.g., slow and shallow breaths, slow breaths to tidal volume, quick and shallow breaths, quick breaths to tidal volume).

During periods of time when the patient is breathing, the method comprises causing airflow into the left nare (block 1002), and causing airflow into the right nare (block 1004). As before, the causing of airflow may be by application of negative pressure to the patient's mouth. The negative pressure applied to the mouth may be between 1 and 12 cmH20 below atmospheric, and more particularly between 6 and 8 cmH2O below atmospheric, but other negative pressures may be used.

The illustrative method further comprises controlling airflow with respect to the patient's mouth (block 1006). Unlike previous embodiments that could only control pressure in the mouth by application of positive pressure, a nasal function test device according to system 700 may selectively applied both positive pressure (e.g., during inhalation by the patient) and negative pressure (e.g., during exhalation by the patient). Thus, overall pressure control within the patient's mouth during respiration may be better than for positive pressure only devices. In one example variation the pressure associated with the airflow of the patient's mouth may be measured by the sensor 254 in the nasal function test device 700, and the blowers 702 and/or 710 adjusted to control pressure to be about constant. In other embodiments, particularly lower cost systems, only a blower configured for application of negative pressure may be present, and thus in such embodiments the pressure control at the patient's mouth may be selective application of negative pressure.

In this ninth example method, the value indicative of nasal resistance is determined based on properties of the airflow into the nares, as well as properties of the airflow associated with the mouth, which again may be controlled. With airflow into the nares (again blocks 1002 and 1004), and controlled airflow with the respect to the mouth (again block 1006), the nasal function test device measures and/or determines one or more properties of the airflow into the nares (block 1008), and one or more properties of airflow associated with the mouth (block 1010). Dashed line 1014 indicates that the control illustrated by block 1006 may be based on the same properties measured, as indicated by block 1010. From the properties a value indicative of nasal resistance of one or both of the nares is determined (block 1012).

Consider, as an example, that the sensors 234 and 244 each comprises an airflow sensor (that measures airflow velocity or rate), and a pressure sensor. Moreover, consider that the sensor 254 associated with the oral airway comprises a pressure sensor. Thus, in one example embodiment the nasal function test device measures airflow into each nare, a pressure of the airflow developed for each nare, and the airflow pressure associated the mouth (and controls based on the measured pressure). From the pressures and airflow, a value indicative of nasal resistance may determined, such as by use of Equation (4) above. In some embodiments, particularly embodiments with lower cost nasal function devices, the nares may be treated as a singular entity, with airflow forced in the both nares by way of a common plenum, and the nasal resistance determined based on a single measured pressure and airflow with respect to the nares; however, while the value indicative of nasal resistance determined with nares considered a single entity may be useful, other information may be lost in such embodiments, such as differences in nasal resistance between the nares.

### Method Embodiment 10

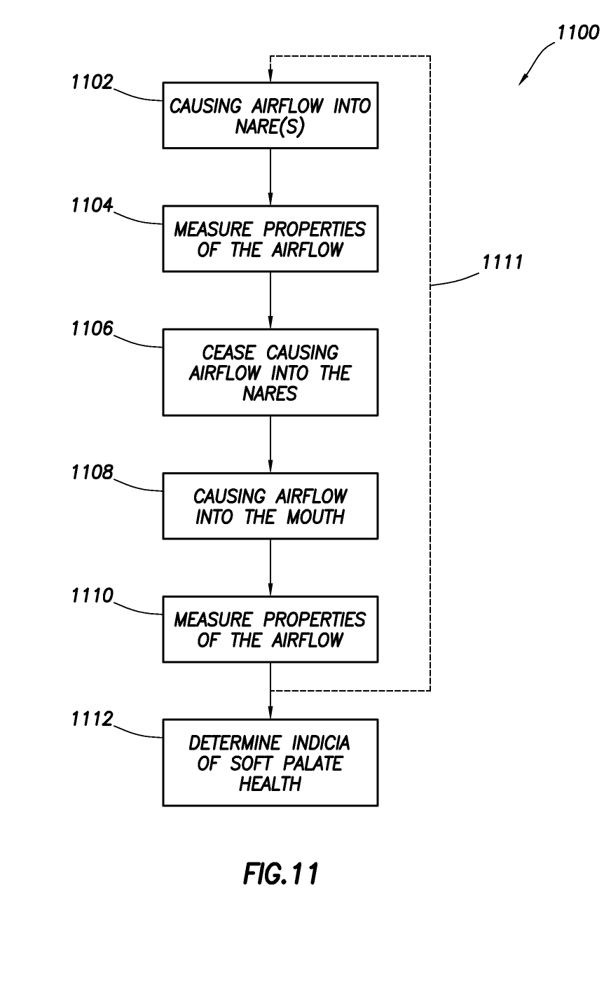

FIG. 11 shows a method 1100 in accordance with at least some embodiments. The illustrative method of FIG. 11 involves determining values indicative of nasal resistance, but in this case associated with the health of the soft palate 32 of the patient. In particular, method 1100 involves causing airflow into one or both nares (block 1102), and measuring a property of the airflow (block 604). In some cases, the causing of airflow may be by application of positive pressure to the nares, and in other cases the causing of airflow may be by application of negative pressure to the mouth, or both. In some cases, the airflow is established before the measurement takes places, and in other cases the establishing and measuring may occur simultaneously. Regardless of the precise time, the time that airflow is provided is sufficient to move or place the soft palate 32 in an open orientation (again, shown in solid lines in FIG. 2). Regardless of the precise predetermined period of time (e.g., up to 1 s in some cases, and up to 5 s in other cases), the causing of airflow eventually ceases (block 1106), and airflow changes direction.

In particular, the method may then comprise causing airflow into the mouth (block 1108) with the goal of forcing the soft palate to close, and measuring a property of the airflow (block 610). In some cases, the causing of airflow into the mouth may be by application of positive pressure to the mouth, and in other cases the causing of airflow into the mouth may be by application of negative pressure to one or both nares, or both. A closed soft palate is shown in dashed lines in FIG. 2 as soft palate 32A. In some cases, the airflow is established before the measuring takes place, and in other cases the measurement may occur simultaneously. As before, airflow into the mouth will flow past the soft palate and out the nares only momentarily, and such flow tends to cause the soft palate to close or block the nasopharynx. Thus, in most patients the reverse flow that exits the nares may be very small, or even nonexistent. A small percentage of patients may be able to sustain the reverse airflow, and this ability too may be indicative of health of the soft palate. In many cases, the forcing of airflow repeats at least once, as illustrated by line 1111.

The method proceeds to determining an indicia of soft palate health (block 1112), which is a component of the overall nasal resistance. The indicia of soft palate heath may take many forms. In some cases, the rise time to establish flow from the nares through mouth after the soft palate has closed (i.e., just after a “reverse” flow”), may be an indication of soft palate health, with rise time indicative of how quickly the soft palate may move to an open orientation. Likewise, the amount of reverse flow before the soft palate closes (i.e., just after a forward flow), and/or the time it takes the soft palate to close after application of a known volume or rate of airflow, may be an indication of soft palate health.

### Method Embodiment 11

Finally, a hybrid method may be used as a transitory test as the industry comes to grips with a system where patient effort can be completely removed from nasal function testing. In particular, while connected to a nasal function test device (e.g., system 200, or system 700), the patient may be asked to breath normally, and the nasal function test device can then measure airflow and/or vacuum created as indication of patient breathing effort. The nasal function test device may then duplicate that patient breathing effort by application of pressure and/or vacuum during periods of time when the patient has paused breathing. Thus, not only then will the nasal function test be at simulated respiratory effort matching or closely matching the normal respiratory effort, but the simulated respiratory effort may be repeatable over time (e.g., hours, days, weeks, months), thus providing repeatability of tests. Moreover, later tests may be made at the previous respiratory effort even if the patient themselves cannot later develop the same effort (e.g., because of age, illness, or surgery).

In like fashion, while connected to a nasal function test device the patient may be asked to breath with maximum effort, and the nasal function test device can then measure airflow and/or vacuum created as indication of patient high breathing effort. The nasal function test device may then duplicate that patient high breathing effort by application of pressure and/or vacuum during periods of time when the patient has paused breathing. Thus, not only then will the nasal function test be at simulated respiratory effort matching or closely matching the previous respiratory effort, but the simulated respiratory effort may be repeatable over time (e.g., hours, days, weeks, months), thus providing repeatability of tests. Moreover, later tests may be made at the previous respiratory effort even if the patient themselves cannot later develop the same high effort (e.g., because of age, illness, or surgery).

Further still, with the various embodiments it is possible to create an “industry standard” breathing effort and/or pattern, and the “industry standard” breathing effort may be applied by the device to each patient during a pause in breathing such that nasal resistance of a particular individual may be measured and then compared across the population, all without patient effort affecting measured results.

In many embodiments, the nasal function test device (e.g., system 200, or system 700) may be coupled to the patient for a very short period of time, for example the amount of time used for a single nasal resistance determination for each nare. However, in other cases the nasal function test device may be coupled to the patient for an extended period of time, for example over night. In such cases, the time varying nature of the patient's nasal resistance may be determined. However, in embodiments where the patient may be coupled to the device for an extended period, the possibility exists that expelled carbon dioxide may build up in the conduits in such a way as to limit the oxygen flowing to the patient. To address such situations, in some embodiments the nasal interfaces and/or oral interface may have an aperture representing a controlled leak for the escape of carbon dioxide. In other cases, the dump valves 233, 243, 253 may be fully or partially opened to enable exhaled carbon dioxide to escape the system. In either case, the controlled leak will represent a known or pre-determined leak, and as such may be compensated for in the determinations of nasal resistance.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent . . . 95 percent, 96 percent, 97 percent, 98 percent, 99 percent, or 100 percent. Moreover, any numerical range defined by two R numbers as defined in the above is also specifically disclosed. Use of broader terms such as “comprises”, “includes”, and “having” should be understood to provide support for narrower terms such as “consisting of”, “consisting essentially of”, and “comprised substantially of”. Accordingly, the scope of protection is not limited by the description set out above but is defined by the claims that follow, that scope including all equivalents of the subject matter of the claims. Each and every claim is incorporated as further disclosure into the specification, and the claims are embodiment(s) of the present invention.

References to “one embodiment”, “an embodiment”, “a particular embodiment”, “some embodiments”, “various embodiments”, and “example embodiments” indicate that a particular element or characteristic is included in at least one embodiment of the invention. Although the illustrative phrases may appear in various places, these do not necessarily refer to the same embodiment.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1-19. (canceled)

20. A method comprising:
applying negative pressure to a patient's mouth and thereby creating airflow that flows in a first nare and out the patient's mouth;
measuring at least one property of the airflow during the causing; and
determining an indicia of nasal resistance of the patient, the determining based on the at least one property.

21. The method of claim 20:
wherein measuring at least one property further comprises measuring airflow rate; and
wherein determining an indicia of nasal resistance further comprises determining the indicia of nasal resistance based on the airflow rate.

22. The method of claim 21:
wherein measuring at least one property further comprises measuring a pressure associated with the airflow into the first nare; and
wherein determining an indicia of nasal resistance further comprises determining the indicia of nasal resistance based on the airflow rate and the pressure.

23. The method of claim 20:
wherein measuring at least one property further comprises:
measuring airflow rate into the first nare;
measuring narial pressure associated with the airflow rate;
measuring oral pressure associated with the patient's mouth; and
wherein determining an indicia of nasal resistance further comprises:
calculating the difference between narial pressure and the oral pressure to create a pressure value; and
dividing the pressure value by the airflow rate.

24. The method of claim 20 further comprising refraining, by the patient, from breathing during the causing and measuring.

25. The method of claim 20 further comprising, during the causing, measuring, and determining, breathing by the patient.

26. The method of claim 20:
wherein causing comprises:
applying negative pressure to the patient's mouth and thereby causing airflow into the first nare; and then
ceasing application of negative pressure; and then
implementing airflow into the patient's mouth and out the first nare;
wherein determining an indicia of nasal resistance further comprises determining the indicia based on properties of airflow during the applying and implementing.

27. The method of claim 26 wherein implementing further comprises at least one selected from the group consisting of: applying pressure to the patient's mouth; and applying negative pressure to the first nare.

28. A system comprising:
a control system comprising a processor and a memory device coupled to the processor;
an airflow system operatively coupled to the control system and fluidly coupled to a first conduit; and
a first sensor fluidly coupled to the first conduit and electrically coupled to the processor, wherein the first sensor is configured to measure a property of the airflow from the airflow system;
wherein the memory storing a program that when executed by the processor causes the processor to:
cause, by way of negative pressure applied by the airflow system, airflow into a first nare of a patient;
measure, by way of the first sensor, at least one property of the airflow; and
determine an indicia of nasal resistance of the patient based on the at least one property.

29. The system of claim 28:
wherein the first conduit is fluidly coupled to a first nare of the patient; and
wherein the first sensor is configured to measure airflow rate;
wherein when the processor measures, the program causes the program to measure a rate of airflow into the first nare by way of the first sensor; and
wherein when the processor determines the indicia of nasal resistance, the program causes the processor to determine the indicia of nasal resistance based on the rate of airflow into the first nare.

30. The system of claim 29 further comprising:
a second sensor operatively coupled to the first conduit and electrically coupled to the processor, the second sensor configured to measure pressure;
wherein when the processor measures, the program further causes the program to measure pressure of airflow into the first nare; and
wherein when the processor determines the indicia of nasal resistance, the program causes the processor to determine the indicia of nasal resistance based on the pressure of the airflow and the rate of the airflow into the first nare.

31. The system of claim 28:
wherein the first conduit is fluidly coupled to a first nare of the patient;
a second conduit having a second patient interface, the second fluidly coupled to the patient's mouth;
a second sensor fluidly coupled to the second conduit and electrically coupled to the processor, wherein the second sensor is configured to measure a property of airflow through the patient's mouth;
wherein when the processor measures, the program further causes the program to measure a property of airflow through the patient's mouth; and
wherein when the processor determines the indicia of nasal resistance, the program causes the processor to determine the indicia of nasal resistance based on the property of airflow into the first nare and the property of the airflow through the patient's mouth.

32. The system of claim 31:
wherein the first sensor is configured to measure airflow rate in the first nare;
wherein the second sensor is configured to measure pressure of airflow within the first conduit;
a third sensor fluidly coupled to the first conduit, the third sensor configured to measure pressure of airflow in the first conduit
wherein when the processor measures, the program causes the program to measure a rate of airflow into the first nare by way of the first sensor, to measure pressure of airflow at the patient's mouth by way of the second sensor, and to measure pressure of airflow at the first nare; and
wherein when the processor determines the indicia of nasal resistance, the program causes the processor to determine the indicia of nasal resistance based on the rate of airflow into the first nare, the pressure of airflow through the patient's mouth, and the pressure of airflow through the first nare.

33. The system of claim 32 wherein when the processor determines the indicia of nasal resistance, the program causes the processor to
calculate a difference between pressure at the first nare and pressure at the patient's mouth, the calculation creates a pressure value; and
divide the pressure value by the airflow rate into the first nare.

34. A diagnostic device comprising:
a processor;
a memory coupled to the processor;
a first blower electrically coupled to the processor;
a first port exposed through an aperture in an exterior surface of the diagnostic device, the first port fluidly coupled to an inlet of the first blower;
a second port exposed through an aperture in the exterior surface of the diagnostic device;
a first sensor fluidly, the first sensor electrically coupled to the processor, and the first sensor configured to measure a property of airflow associated with the first blower;
wherein the memory storing a program that, when executed by the processor, causes the processor to:
cause, by way of the first blower, airflow into a first nare of a patient;
measure, by way of the first sensor, at least one property of the airflow; and
determine an indicia of nasal resistance of the patient based on the at least one property.

35. The diagnostic device of claim 34 further comprising:
a second blower, an inlet of the second blower fluidly coupled to the inlet of first blower, and an outlet of the second blower fluidly coupled to the first port;
wherein program further causes the processor to:
force, by way of the second blower, airflow into the a mouth of the patient; and
measure, by way of the first sensor, at least one property of airflow associated with the first nare;
wherein when the processor determines the indicia of nasal resistance, the program causes the processor to determine based on airflow caused by the first blower and airflow caused by the second blower.

36. The diagnostic device of claim 34:
wherein the first sensor is configured to measure airflow rate;
wherein when the processor measures, the program causes the processor to measure a rate of airflow by way of the first sensor; and
wherein when the processor determines the indicia of nasal resistance, the program causes the processor to determine the indicia of nasal resistance based on the rate of airflow.

37. The diagnostic device of claim 36 further comprising:
a second sensor fluidly coupled between the second blower and the first port, the second sensor electrically coupled to the processor, and the second sensor configured to measure pressure;
wherein when the processor measures, the program further causes the processor to measure a pressure of airflow provided to the first port; and
wherein when the processor determines the indicia of nasal resistance, the program causes the processor to determine the indicia of nasal resistance based on the pressure of the airflow and the rate of the airflow.

38. The diagnostic device of claim 37:
a third sensor fluidly coupled a second port, the third sensor electrically coupled to the processor, and the second sensor configured to measure a pressure;
wherein when the processor measures, the program further causes the processor to measure pressure of airflow associated with the second port; and
wherein when the processor determines the indicia of nasal resistance, the program causes the processor to determine the indicia of nasal resistance based on pressure of airflow associated with the first port, pressure of airflow associated with the second port, and airflow associated with the first nare.
